(19) United States
(12) Patent Application Publication
(10) Pub. No.: US 2009/0226575 A1
(43) Pub. Date: Sep. 10, 2009

(54) Low glycemic sweetener compositions

(76) Inventors: Stephen B. Roman, Phoenix, AZ (US); Gary S. Kehoe, Phoenix, AZ (US)

Correspondence Address: TOD R NISSLE, PO BOX 55630, PHOENIX, AZ, 85078, US

(21) Appl. No.: 12/319,288

(22) Filed: Oct. 18, 2007

Related U.S. Application Data
(63) Continuation-in-part of application No. 11/900,783, filed on Sep. 12, 2007.
(60) Provisional application No. 60/852,945, filed on Oct. 19, 2006.
(60) Provisional application No. 60/994,004, filed on Sep. 17, 2007.

(51) Int. Cl. G01N 33/02

(52) U.S. Cl. 426/231

(57) ABSTRACT

A method is provided to sweeten a food product to minimize the glycemic load of the food product, to introduce natural fiber into the food product, to produce a desired sweetness profile, and to reduce the likelihood that the food product when ingested causes gastrointestinal distress. The method includes the steps of selecting at least one polyol; determining the laxation threshold in grams per day of the polyol(s); determining the desired quantity of a sweetener composition in a serving of the food product, the sweetener composition comprising the polyol(s) and agave syrup solids added in solid form during the manufacture of the food product; determining the desired sweetener profile of the sweetener composition, the sweetener composition including proportions of agave and the polyol(s) to minimize the likelihood the sweetener composition will cause the food product to cause gastrointestinal distress; the adjustment of sweetness by adding a high intensity sweetener(s); admixing the sweetener particles during the manufacture of the food product to produce the desired quantity of the sweetener composition in a serving of the food product.

This application claims priority based on provisional application Ser. No. 60/852,945 filed Oct. 19, 2006, and is a continuation-in-part of patent application Ser. No. 11/900,783, filed Sep. 12, 2007, and its associated provisional application 60/994,004, filed Sep. 12, 2006.

This invention relates to sweetener compositions.

More particularly, the invention relates to sweetener compositions that control the glycemic load of a food product, that introduce natural fiber into the food product, that produce a desired sweetness profile, and that reduce the likelihood that the food product when ingested causes gastrointestinal distress.

In a further respect, the invention relates to sweetener compositions that release from a food product, a polyol and agave syrup when the food product is in the digestive tract of an individual.

The use of polyols in food products in combination with various sweeteners is well known. The sweeteners are added to an already manufactured polyol, or are added during the manufacture of a food product as an ingredient separate from the polyol. Polyols have a lower glycemic load than sucrose or high fructose corn syrup, and, as do a variety of other substances, have a lower laxation threshold than sucrose or high fructose corn syrup.

We have discovered a sweetener composition and method that enables, simply by adding the sweetener composition during manufacture of the product a food product, the achievement of a desired sweetness profile in the food product for a polyol and associated sweetener, of a desired glycemic load, of an acceptable gastrointestinal tolerance level, and of the release of a polyol and associated sweetener in the digestive tract of an individual that consumes one or more servings of the food product.

The method includes the steps of selecting at least one polyol; determining the laxation threshold in grams per day of the polyol(s); determining the desired quantity of a sweetener composition in a serving of the food product, the sweetener composition comprising the polyol(s) and agave added in solid or liquid form during the manufacture of the food product; determining the desired sweetener profile of the sweetener composition, the sweetener composition including proportions of agave and the polyol(s) to minimize the likelihood the sweetener composition will cause the food product to cause gastrointestinal distress; the adjustment of sweetness by adding a high intensity sweetener(s); admixing the polyol, agave and high intensity sweetener in powder form to produce a sweetener mixture, or admixing the polyol, agave and high intensity sweetener(s) in liquid form to produce a sweetener mixture; drying and fractioning the sweetener mixture to produce sweetener particles combined with agave; and, admixing the sweetener particles during the manufacture of the food product to produce the desired quantity of the sweetener composition in a serving of the food product.

The polyol selected can, by way of example and not limitation, comprise sorbitol, mannitol, polyglycitol syrup, polyglycitol powder, maltitol syrup, maltitol, xylitol, lactitol, isomalt, and erythritol. The glycemic load of these polyols is low in comparison to sucrose. The laxation threshold in grams/day of these polyols has been reported in technical literature as sorbitol (50), mannitol (20), polyglycitol syrup (greater than 100), polyglycitol powder (greater than 150), maltitol syrup (greater than 100), malititol (60-90) xylitol (50-90), lactitol (20-50), isomalt (50-70), and erythritol (125). The % relative sweetness of these polyols with respect to sugar has been reported in technical literature as sorbitol (60), mannitol (50), polyglycitol syrup (30-40), polyglycitol powder (less than 20), maltitol syrup (70-80), malititol (90) xylitol (100), lactitol (30-40), isomalt (40), and erythritol (60-70). Agave syrup can be as high as 120% as sweet as sucrose. In addition, the laxation threshold of agave syrup is less than most, if not all, polyols.

The desired quantity of sweetener in a food product will, of course, depend on the food product. A dessert-type food product like cake or an apple turnover will require a higher weight percent of a traditional sweetener such as sucrose, than a less sweet food product like bread. Regardless of the food product, however, the quantity of sweetener in a serving of the food product can be readily determined. This is evidenced by the fact that on most food products the serving size and quantity of sugar or other sweeteners is typically set forth on a label on the food product. The desired amount of sucrose or other sweetener utilized in the product is also readily determined either by past experience in making the produce or by current formulation of a desired product. If, for example, fifty grams of sucrose is required in a food product, then the desired quantity of a polyol, a high intensity sweetener(s) and an associated sweetener to provide an equivalent sweetness is determined by considering the serving size, by considering the laxation threshold and percent relative sweetness of the polyol, and by considering the laxation threshold and the percent relative sweetness of the sweetener associated with the polyol.

First, for sake of discussion, assume that the polyol selected is just as sweet as sucrose. Then, if for example the polyol is sorbitol and the sweetener is agave syrup, the laxation threshold of sorbitol is 50 grams/day. If the serving size is one, i.e., the food product likely will be entirely consumed in one sitting, then fifty grams of sorbitol is not utilized in the product because it is likely the product would have a laxative effect. Less than fifty grams of sorbitol is utilized in the product. For example, thirty grams of sorbitol and twenty grams of agave syrup are utilized to insure that the food product likely will not have a laxative effect. If, on the other hand, the food product includes ten servings and it is unlikely that more than a couple servings will be consumed in a day, then fifty grams of sorbitol likely could be used because only five grams of sorbitol is consumed in each serving. As a general rule, it might well be preferred to limit the amount of sorbitol to eight to ten grams per serving to reduce the risk that sufficient sorbitol will be ingested to cause laxation, although such limitations can be set as desired. High intensity sweeteners allow for such an adjustment, without reducing the target level of sweetness desired in the final product. They are therefore used to help balance between gastrointestinal effects caused by higher levels of polyols potentially used in food formulations, and desired sweetness in the food product.

Second, for sake of further discussion, it is noted that sorbitol is only 60% as sweet as sucrose. If the sweetness provided by 50 grams of sucrose is desired in the food product, either more than fifty grams of sorbitol has to be utilized, or the sorbitol has to be combined with or replaced in part by a polyol or sweetener that has a greater percent relative sweetness than does sorbitol. Consequently, a high intensity sweetener like aspartame or a natural high intensity sweetener such as stevia or rebaudioside A can be utilized, as can agave syrup which can be as high as 120% the sweetness level of sugar. Agave syrup is a recommended sweetener in the practice of the invention because it can include soluble inulin fiber up to 100%.

A food product like gum may well be able to utilize a sweetener that is 100% by weight sorbitol and high intensity sweetener(s) because the serving size is small. Other food products like cakes (or the cake mix used to make the cake), or ice cream are eaten in larger serving sizes which will include a greater quantity of sweetener and which may well, to avoid a laxation effect, require that other sweeteners be utilized in combination with the polyol.

In sum, as noted in the discussion above, after a desired quantity of sucrose (or another sweetener) in a food product and/or a serving of a food product is determined, a determination is made of the proportion of agave syrup or other sweetener such as a natural or artificial high intensity sweetener(s) and polyol necessary to provide an equivalent sweetness and to minimize the likelihood that the polyol in the food product will produce a laxation effect when the food product is consumed.

In accordance with the invention, a sweetener can be produced by infusing the polyol with agave syrup and a natural or artificial high intensity sweetener(s). This is accomplished by admixing liquid polyol, high intensity sweetener(s) and liquid agave syrup to produce a sweetener mixture. This mixture can be dried and fractioned to produce sweetener particles. The proportion of sorbitol, high intensity sweetener(s) and agave in the sweetener particles is sufficient to produce a relative sweetness that preferably is, when a weight of sweetener particles equivalent to the weight of sucrose (or of high fructose corn syrup or other sweetener(s)) utilized in a food product, equivalent to within plus or minus five percent of the relative sweetness provided by an equivalent weight of sucrose.

### EXAMPLE I

The following ingredients are provided:

| | | |
|---|---|---|
| Polyglycitol syrup (or other polyol(s)) | 40% | (w/w) |
| Agave syrup (or other sweetener(s)) | 59.10% | (w/w) |
| Water | 0.75% | (w/w) |
| Stevia, high intensity sweetener | 0.15% | (w/w) |

Solid agave-combined polyol sweetener particles are produced as follows:
(a) The water is added to a mixing vessel.
(b) The polyglycitol is admixed with the water to produce a water-polyglycitol composition.
(c) The agave syrup, and then the stevia, is admixed with the water-polyglycitol composition to produce a sweetener composition. Further desired additives and ingredients including the high intensity sweetener(s) may, if desired, be added at this stage and admixed to produce a uniform composition.
(d) The sweetener composition is heated to 330 degrees F. and subjected to a vacuum of 24-27 in/Hg while under agitation.
(e) The heat and vacuum of (d) are maintained until the moisture level in the sweetener composition is below 0.75% by weight. Further desired additives and ingredients including the high intensity sweetener(s) may be added at this stage and admixed to produce a more uniform composition.
(f) The cooked mass is aerated to improve appearance and fracturing during subsequent milling.
(g) The cooked mass is formed into a rope via an extrusion process and the extrudate is then sized using a Fitz mill or other fractioning classifier to yield particles of a desired size. Further desired additives and ingredients may be added during this fractioning/classifying process.

The foregoing process can be carried out by a batch process, a continuous process, or a combination of the two. The order of addition and combination of the ingredients can be varied as desired depending on the types of ingredients being blended and the respective physical and/or chemical characteristics of the ingredients.

Examples of processes that may be utilized during the Example 1 process include, without limitation, are drum crystallization, spray drying, extrusion, fluid bed drying, dry blending/agglomeration, roll drying and block crystallization equipment.

### EXAMPLE 2

The following ingredients are provided:

| | | |
|---|---|---|
| Maltitol solid particulate (or other polyol(s)) | 45% | (w/w) |
| Agave syrup (or other sweetener(s)) | 45% | (w/w) |
| Water | 9.85% | (w/w) |
| Stevia (Stevioside/rebaudioside-A) | 0.15% | (w/w) |

An agave-combined polyol liquid composition is produced as follows:
(a) The water is added to a mixing vessel.
(b) The maltitol is admixed with the water to produce a water-maltitol composition.
(c) The agave syrup, and then the stevia, are admixed to uniformity with the water-maltitol composition to produce a sweetener composition. Further desired additives and ingredients including high intensity sweetener(s) may, if desired, be added at this stage and admixed to produce a uniform composition.

In Examples 1 and 2, other ingredients that may be added include fibers, flavors (like vanilla, for example), aromatics, soluble fiber, acids, extracts, fruit concentrates, fructose (and other sugars), syrups, minerals, mineral complexes, and appetite suppressants. Examples of other sweeteners include honey and brown rice syrup, among others. Examples of high intensity sweeteners include aspartame, acesulfame-K, sucralose, lo han guo, stevia, rebaudioside A, thaumatin, neohesperidin, dihydrochalcone, and monatin.

The weight percent of a polyol(s) in the solid or liquid compositions produced in Examples 1 and 2 is in the range of 10% to 80% by weight, as is the weight percent of agave syrup (liquid composition) or agave syrup solids (solid composition). When high intensity sweeteners are utilized in the solid or liquid compositions, it is in at a weight percent in the range of 0.0001% to 3.5% by weight.

In one embodiment of the invention, the sweetener-combined polyol liquid compositions presently include 26% to 75% by weight polyol, 15% to 65% by weight natural sweetener (such as agave syrup), and 5% to 45% by weight water. The liquid compositions can also include 0.0001% to 3.5% by weight of a high potency sweetener such as stevia/rebaudioside A.

In another presently preferred embodiment of the invention, the sweetener-combined polyol liquid compositions presently include 46% to 56% by weight polyol, 44% to 54% by weight natural sweetener (such as agave syrup), and 15% to 25% by weight water. The liquid compositions can also include 0.01% to 3.5% by weight of a high potency sweetener such as stevia/rebaudioside A.

In another embodiment of the invention, the sweetener-combined polyol solid compositions presently include 36% to 85% by weight polyol, 15% to 64% by weight natural sweetener (such as agave syrup solids), and 0% to 5% by weight water. The liquid compositions can also include 0.0001 % to 3.5% by weight of a high potency sweetener such as stevia/rebaudioside A.

In a further presently preferred embodiment of the invention, the sweetener-combined polyol solid compositions presently include 56% to 66% by weight polyol, 34% to 44% by weight natural sweetener (such as agave syrup solids), and 0.2% to 1.5% by weight water. The liquid compositions can also include 0.01% to 3.5% by weight of a high potency sweetener such as stevia/rebaudioside A.

Any desired polyol(s) can be utilized in producing the sweetener-combined polyol solid and liquid compositions of the invention. Examples of polyols includes, without limitation, maltitol, sorbitol, isomalt, polyglycitols, xylitol, erythritol, lactitol, mannitol, SORBOGEM and SORBO by Corn Products Specialty Ingredients, NEOSORB by Roquette, SORBITOL SOLUTION and CRYSTALLINE SORBITOL by Archer Daniels Midland (ADM), SORBIDEX by Cargill, MALTISWEET, STABILITE and HYSTAR by Corn Products Specialty Ingredients, MALTISORB and LYCASIN by Roquette, MALTIDEX and MALBIT by Cargill, LESYS-T and AMALTY by Towa Chemical Industry Col, Ltd.

Agave Syrup may be sourced from one or more, without limitation, of the following companies: Nekutli, IIDEA, AG Commodities, Sweet Cactus Farms, Blue Agave Syrup, and American Health and Nutrition.

The water utilized in the practice of the invention may be de-ionized water, USP water, de-chlorinated water, mineral water, treated water, or tap water. The preferred water is deionized water.

Stevia Rebaudiana may be sourced from one or more, but not limited to the following companies: AIDP, Inc.; Alfa Chem; Ama Nutrasource Inc.; American Ingredients; AnMar Nutrition; AsiaAmerica Intl, Inc.; Draco Natural Products; Falcon Trading Intl. GCI Nutrients; Functional Foods Corp.; Herb Trade Inc.; Morita Kagaku Kogyo Co.; Ltd.; Motherland Intl.; NuNaturals, Inc.; Nutriland Group, Inc.; Nutrichem Resources Co.; NutriScience Innovations LLC; Pharmachem Laboratories, Inc.; Pharmline Inc.; Ria Intl LLC; San Francisco Herb & Natural Food Co.; Starwest Botanicals; Stauber Performance Ingredients; Strategic Sourcing, Inc.; Stryka Otanics; P.O. Thomas & Co., Inc.; Watson Industries; and, Wright Group.

Lo han guo examples include PURELO by Bio Vittoria, MORMORDICA by Draco Natural Products, lo han guo concentrate by Amax Nutrasource.

Any source of soluble, insoluble and/or fermentable fiber(s), gums, hydrocolloids and resistant starch derived materials can be utilized in the compositions of the invention including, by way of example and not limitation, mucilages, hemicellulose, cellulose, lignin, psyllium seed husk, beta-glucan, pectins, gum arabic, oat bran, locust bean gum, fructans, xanthan gum, fructooligosaccharides, oligosaccharides, polysaccharides, resistant starches, polydextrose, inulin, arabinogalactan, hydrolyzed guar gum, alginates, tragacanth, arabinoxylan, and resistant maltodextrins by way of example and not limitation, NUTRIOSE™ by Roquette, and FIBERSOL™ by Matsutani Chemical Industry Col.

Any natural sweetener(s), appetite suppressant(s), and/or high potency sweetener(s) may be used in the compositions of the invention including, by way of example and not limitation, raw organic honey, maple syrup, inulin, barley malt, brown rice syrup, granulated brown rice sweetener, fruit concentrate, fruit juice sweeteners, organic molasses, sorghum syrup, date sugar, organic dried natural cane juice and trehalose.

As used herein, a polyol is a hydrogenated form of carbohydrate whose carbonyl group (aldehyde or ketone, reducing sugar) has been reduced to a primary or secondary hydroxyl group.

As used herein, a combined polyol liquid or solid is a polyol in which a sweetener or other component or composition is generally homogeneously dispersed throughout the polyol liquid or solid.

As used herein, a solid polyol particle contains at least 36% to 85% by weight of one or more polyols.

As used herein, a solid polyol particle combined with a sweetener contains 36% to 85% by weight polyol and 15% to 64% by weight sweetener.

As used herein, a sweetener is any natural or synthetic sweetener including, but not limited to, natural and synthetic high intensity sweeteners such as lo han guo, stevia, rebaudioside A, acesulfame potassium, thaumatin, glycyrrhizin, neohesperidin dihydrochalcone, aspartame, saccharin and sucralose.

As used herein, a polyol liquid contains 26% to 75% by weight polyol, 15% to 65% by weight sweetener, and 5% to 45% by weight water.

As used herein, a solid bulk sweetener-polyol composition consists of sweetener-combined polyol particles shipped in a container holding at least fifty pounds of the sweetener combined particles.

As used herein, a liquid bulk sweetener is a sweetener-combined liquid polyol composition shipped in a container holding at least forty pounds of the sweetener-combined liquid polyol composition.

Having described our invention in such terms as to enable it to be made and used by those of skill in the art,

We claim:

1. A method to sweeten a food product to minimize the glycemic load of the food product, to introduce natural fiber into the food product, to produce a desired sweetness profile, and to reduce the likelihood that the food product when ingested causes gastrointestinal distress, said method comprising the steps of
(a) selecting at least one polyol;
(b) determining the laxation threshhold in grams per day of said polyol(s);
(c) determining the desired quantity of a sweetener composition in a serving of the food product, said sweetener composition comprising said polyol(s) and agave syrup solids added in solid form during the manufacture of the food product;
(d) determining the desired sweetener profile of said sweetener composition, said sweetener composition including proportions of agave, and said polyol(s) to minimize the likelihood said sweetener composition will cause the food product to cause gastrointestinal distress;
(e) admixing said polyol(s) and agave syrup to produce a sweetener mixture;
(f) drying and fractioning said sweetener mixture to produce solid sweetener particles combined with agave syrup solids;
(g) admixing said sweetener particles during the manufacture of the food product to produce said desired quantity of said sweetener composition in a serving of the food product.

2. A method to sweeten a food product to minimize the glycemic load of the food product, to introduce natural fiber into the food product, to produce a desired sweetness profile, and to reduce the likelihood that the food product when ingested causes gastrointestinal distress, said method comprising the steps of
(a) selecting at least one polyol;
(b) determining the laxation threshhold in grams per day of said polyol(s);
(c) determining the desired quantity of a sweetener composition in a serving of the food product, said sweetener composition comprising said polyol(s) and agave syrup solids added in solid form during the manufacture of the food product;
(d) determining the desired sweetener profile of said sweetener composition, said sweetener composition including proportions of agave, and said polyol(s) to minimize the likelihood said sweetener composition will cause the food product to cause gastrointestinal distress;
(e) admixing said polyol(s) and agave syrup to produce a sweetener mixture;
(f) drying and fractioning said sweetener mixture to produce solid sweetener particles combined with agave syrup solids;
(g) admixing said sweetener particles during the manufacture of the food product to produce said desired quantity of said sweetener composition in a serving of the food product.

3. The method of claim 1 wherein in step (e) a selected amount of a high intensity sweetener is admixed with said polyol(s) and agave syrup.

4. The method of claim 2 where in step (e) a selected amount of a high intensity sweetener is admixed with said polyol(s) and agave syrup.
